(19) United States
(12) Patent Application Publication
(10) Pub. No.: US 2025/0136799 A1
(43) Pub. Date: May 1, 2025

(54) ETHYLENE SCAVENGING COMPOSITIONS WITH ANTIMICROBIAL PROPERTIES

(71) Applicant: Chicora Group, LLC, Greenville, SC (US)

(72) Inventors: Ross Lyon, Greenville, SC (US); Mark Szarka, Greenville, SC (US)

(73) Assignee: Chicora Group, LLC, Greenville, SC (US)

(21) Appl. No.: 18/836,165

(22) PCT Filed: Sep. 30, 2022
(86) PCT No.: PCT/US2022/045314

§ 371 Date: Aug. 6, 2024

(51) Int. Cl. C08L 23/12; A23B 2/725; C08K 3/015; C08K 5/00

(52) U.S. Cl. CPC C08L 23/12; C08K 3/015; C08K 5/0058; A23B 2/725

(57) ABSTRACT

Disclosed are various embodiments for an ethylene scavenging composition with anti-microbial properties that may be applied to a substrate that includes at least one layer of the ethylene scavenging composition and at least one layer of substrate. The composition includes a base material and at least one active additive. The at least one layer of the ethylene scavenging composition is applied to the at least one layer of substrate.

### FIELD

This invention relates to a composition with high ethylene scavenging ability with antimicrobial properties, and more generally relates to a composition with high ethylene scavenging ability that includes a base material, such as polyester, with an active additive, such as an ethylene scavenging agent and antimicrobial agent for application to a storage container, such as a corrugated box used to transport vegetables and fruit to extend the shelf life.

### BACKGROUND

Over centuries supply chains have evolved to support a constantly expanding global population and growing list of consumer demands. In the case of food items, such as produce, and other flora, the supply chains have adapted but the goods themselves remain limited in their durability. For as long as farmers have been shipping produce, they have had to deal with the issue of spoilage. Despite advances in refrigeration and packaging design, shelf life of produce is still capped, and product waste/spoilage remains a problem. One estimate believes that close to 40% of all harvested produce spoils before reaching the customer. This is just harvested produce. A large amount (greater than 15%) of produce is grown, but not harvested, due to being overly ripe. The primary reasons for this spoilage are the ethylene feedback loop and the presence of bacteria and fungi which can stress and weaken produce. The ethylene feedback loop occurs due the presence of ethylene gas. This gas is released by plants as they ripen. In addition, the presence of ethylene serves as a signal for the plants to ripen themselves, which in turn creates more ethylene.

There are a number of existing products on the market that seek to achieve similar goals of extending the shelf life of produce. These include (but are not limited to) ethylene absorbing/scavenging sachets, gas treatments, and modified-atmosphere packaging. The goal for all these products is roughly the same: to reduce the ethylene feedback loop and extend the shelf-life of produce. However, many of these existing options have downsides, ranging from effectiveness, ease of use, impact on the taste of the finished product, and, in some cases, issues with toxicity.

It is an object of the present invention to reduce or eliminate the issues of product spoilage in the supply chain, while at the same time, improving the functionality, safety, and ease of use compared to alternatives in the market. Additionally, it is another object of the present invention to allow farmers to harvest produce that had been previously left in the field due to ripeness. This should be a major benefit for all stakeholders, both in terms of profitability and improving the availability of produce.

### SUMMARY

### Technical Embodiments and Technical Discussion

The present invention differs both in functionality and application from existing prior art. The present invention contains an antimicrobial agent, in addition to an ethylene scavenging agent, which not only eliminates the issue of the ethylene feed-back loop and premature ripening, but also, reduces the impact of various fungi and microbes which have a detrimental impact on the freshness of produce to extend shelf life. The improvements to the present invention allow a packer to reduce the work required at the packing facility to prevent premature ripening and allow for a fresher product arriving at the desired location. The present invention may consist of a layered structure, comprising at least one layer of a base material with an ethylene scavenging agent and an antimicrobial agent applied to at least one layer of a substrate.

### BRIEF DESCRIPTION OF THE DRAWINGS

Many aspects of the present disclosure can be better understood with reference to the following drawings. The components in the drawings are not necessarily to scale, with emphasis instead being placed upon clearly illustrating the principles of the disclosure. Moreover, in the drawings, like reference numerals designate corresponding parts throughout the several views.

FIG. 1 is a perspective view of a layered structure according to the present invention comprising a layer of composition applied to a layer of substrate; and

FIG. 2 is an exploded view of a layered structure according to the present invention comprising a layer of composition applied to a layer of substrate.

### DETAILED DESCRIPTION

In the following detailed description, reference is made to the accompanying figures, which form a part hereof. In the figures, similar symbols typically identify similar components, unless context dictates otherwise. The illustrative embodiments described in the detailed description, figures, and claims are not meant to be limiting. Other embodiments may be utilized, and other changes may be made, without departing from the scope of the subject matter presented herein. It will be readily understood that the aspects of the present disclosure, as generally described herein, and illustrated in the figures, can be arranged, substituted, combined, separated, and designed in a wide variety of different configurations, all of which are explicitly contemplated herein.

Disjunctive language such as the phrase “at least one of X, Y, or Z,” unless specifically stated otherwise, is otherwise understood with the context as used in general to present that an item, term, etc., may be either X, Y, or Z, or any combination thereof (e.g., X, Y, and/or Z). Thus, such disjunctive language is not generally intended to, and should not, imply that certain embodiments require at least one of X, at least one of Y, or at least one of Z to each be present.

The ranges set forth herein include both numbers at the end of each range and any conceivable number there between, as that is the very definition of a range. It is therefore to be understood that the ranges and limits mentioned herein include all ranges located within the prescribed limits (i.e., subranges). For instance, a range from about 100 to about 200 also includes ranges from 110 to 150, 170 to 190, 153 to 162, and 145.3 to 149.6. Further, a limit of up to about 7 also includes a limit of up to about 5, up to 3, and up to about 4.5, as well as ranges within the limit, such as from about 1 to about 5, and from about 3.2 to about 6.5 as examples.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present invention.

As used herein, the terms “comprise”, “comprises”, “containing”; and “has”, “have”, “having”; and “includes”, “include” and “including”; are open-ended transition terms used to transition from a subject recited before the term to one or more elements recited after the term, where the element or elements listed after the transition term are not necessarily the only elements that make up the subject.

The term weight percent or wt. % means the weight of a given raw material relative to the weight of the resulting composition which includes the raw material. For example, a composition having 55 wt. % of base material means that the composition includes 55 parts by weight of the raw base material relative to 100 parts of the total weight of the resulting composition. It is understood that some residues in the resulting composition differ chemically from the raw material in that the residues in the resulting composition may lack the functional groups at the ends of the respective chains, depending upon the raw material, and the ends of the chains may be bonded.

The present invention includes a base material. The base material may be composed of at least one polymer, such as LLDPE, MLDPE, Metallocene, LDPE, HDPE, Polyolefin, polyethylene, PVdC, PVC, EVOH, polylactic acid (PLA), Nylon (6, 6,6, or some other derivative), polyethylene terephthalate (PET), Polyester, Polypropylene, biopolymer, acrylate polymer, styrene butadienes, PVOH, and/or vinyl acetate. The base material may also include amylum (starch), Metallocenes, or other water based resins, emulsions, adhesives, or binders that are dried to over 80% solids in the finished product may be utilized as the base material. The preferred polymer utilized as the base material is polypropylene, such as ExxonMobil™ pp 3155E5 (a 36 g/10 min MFR (ASTM D1238-13, 2.16 kg, 230° C.) homopolymer, available from ExxonMobil Chemical Company)

The base material may also comprise at least one active additive, and optionally, at least one inactive additive. The active additive may be an ethylene scavenging agent and/or an antimicrobial agent. Preferably, the base material contains both an ethylene scavenging agent and an antimicrobial agent. The active additive is incorporated into the base material to provide additional properties that correspond with the properties of the additive forming a composition.

The ethylene scavenging agent may be incorporated into the base material to form a composition with a high ethylene scavenging capacity for use with food items, such as vegetables and fruits. The composition including the base material and the ethylene scavenging agent may be formed into an article, such as, but not limited to, a film or sheet, resulting in an article with high ethylene scavenging capacity. Additionally, the composition may be extruded into a film or sheet and applied as a laminate to a substrate, such as, but not limited to paper, paperboard, a corrugated box, a carton, and the like, to impart high ethylene scavenging capacity to the substrate. The composition may be applied to the substrate by methods known to those skilled in the art, for example by spray coating, roll coating, knife over roll coating, slot die coating, etc. The surface of the substrate may be treated prior to applying the composition to promote adhesion to the substrate. After the composition is applied to the substrate, it may then be cured in ambient air at ambient temperature, at an elevated temperature, or if the composition is UV activated, subjecting the composition to irradiation with UV light or by exposure to natural light. In a preferred embodiment, the composition is extruded into a film or sheet and applied to an interior portion of a corrugated box or carton, used to store and transport vegetables or fruit.

Examples of ethylene scavenging agents include, but are not limited to, potassium permanganate, zeolites, potassium permanganate-impregnated alumina pellets, activated carbon, activated carbon+Pd-catalyst, activated carbon+bromine type inorganic compounds, and zeolites. A commercially available ethylene scavenging agent is CESA-absorb 19133 PRA0698710 (polyolefin absorber masterbatch) available from Avient Corporation in Winchester, Virginia. The ethylene scavenging agent may be supplied in the form of a thermoplastic concentrate with ethylene scavenging properties. These concentrates (also called “masterbatches”), upon blending with the base material, such as a polypropylene, allow the resultant mixture to consume or scavenge ethylene. The preferred thermoplastic is polyolefin, and the thermoplastic (polyolefin) concentrate is preferably in a pelletized form.

Antimicrobial agent as used herein means a biologically active material that affects the life processes (e.g., inhibits or prevents the implantation, growth and/or life) of a living micro-organism (e.g., algae, fungus, mildew, mold, bacteria). The composition of the present invention contains an effective amount of antimicrobial agent. Effective amount means an amount effective to achieve a desired effect on the living micro-organism. The amount that constitutes an effective amount varies according to the particular antimicrobial agent being employed, the desired effect on the micro-organism, the desired duration of contact, the release rate, and the surface area and location where the antimicrobial agent is to be placed. Accordingly, it is not practical to enumerate particularly preferred amounts but such can be readily determined by those skilled in the art with due consideration of these and other appropriate factors. A commercially available antimicrobial agent is CESAR-am PE J18712-10 PRA0698712 (polyolefin masterbatch containing antimicrobial) available from Avient Corporation in Winchester, Virginia. The antimicrobial agent may be supplied in the form of a thermoplastic concentrate with antimicrobial properties. These concentrates (also called “masterbatches”), upon blending with the base material, such as a polypropylene, allow the resultant mixture to kill or inhibit growth of living microorganisms, such as fungus, bacteria, mold, and bacteria. The preferred thermoplastic is polyolefin, and the thermoplastic (polyolefin) concentrate is preferably in a pelletized form.

The antimicrobial agent may include metal oxides such as copper oxide, silver oxide, and zinc oxide; other copper salts such as copper chlorides, and copper sulfides; metal powders such as copper, zinc, silver, and tin; powders of metal alloys such as of copper, lead, silver, tin, zinc, and mercury; slightly soluble copper compounds such as cupric stearate, cuprous cyanide, and cuprous mercuric iodide; 4,5-dicholor-2-n-octyl-4 isothiazolin-3-one; quaternary ammonium salts based antimicrobial materials such as Dow Corning 5700 from Dow Corning Corp of Midland, Mich., and Nopcocide™ N-96 from Henkel Corp. of Ambler, Pa.; pyrithione zinc 1-octen-3-one, 3-octanol, ethyl pyruvate, propionic acid, trans-3-octen-2-one, chlorine dioxide, and essential oils, fragrance and flavor compounds that can be placed on carriers such as silica gel, zeolite, or kaolin clay and the like.

Inactive additives do not have ethylene scavenging properties or antimicrobial properties. Inactive ingredients can include adhesion promotion agents, pigments, minerals, colorants, dyes, plasticizers, chain extenders and impact modifiers. Examples of typical commercially available impact modifiers well known in the art and useful in this invention include, but are not limited to, ethylene/propylene terpolymers, functionalized polyolefins such as those containing methyl acrylate and/or glycidyl methacrylate, styrene-based block copolymeric impact modifiers, and various acrylic core/shell type impact modifiers. Examples of chain extenders include, but are not limited to, multifunctional (including, but not limited to, bifunctional) isocyanates, multifunctional epoxides, including for example, epoxylated novolacs, and phenoxy resins. Residues of such additives are also contemplated as part of the composition.

The composition of the present invention preferably contains:
from about 19 wt. % to about 99.9 wt. % base material, preferably from about 70 wt. % to about 95 wt. % base material, and most preferably from about 80 wt. % to about 92 wt. % base material;
from about 1 wt. % to about 30 wt. % ethylene scavenging agent, more preferably from about 3 wt. % to about 20 wt. % ethylene scavenging agent, and most preferably from about 5 wt. % to about 15 wt. % ethylene scavenging agent;
from about 0 wt. % to about 10 wt. % antimicrobial agent, from about 0.1 wt. % to about 6 wt. % antimicrobial agent, and most preferably from about 0.1 wt. % to about 3 wt. % antimicrobial agent, wherein the total weight percent of the composition is 100 wt. %.

Preferably, the composition of the present invention is thermoplastic. The form of the composition is not limited and can include a composition in the melt phase polymerization, as an amorphous pellet, as a solid state polymer, as a semi-crystalline particle, as a composition of matter in a melt extrusion zone, as a sheet or film, as a bead, and as a powder. The form of particles formed by the composition are not critical, and such particles may be chips, pellets, flakes, or shredded from a larger material such as sheet or film.

The preferred method of incorporating the active additives and inactive additives into the base material is by solid state blending. In this methods, beads or pellets of the base material, along with beads or pellets of the active additives and any optional inactive additives are added to a vessel and blended together, forming a composition, which is then heated within the vessel and extruded from the vessel.

Alternatively, the composition can be prepared by polymerization procedures known in the art sufficient to effect esterification and polycondensation. Polyester melt phase manufacturing processes include direct condensation of a dicarboxylic acid with the diol, optionally in the presence of esterification catalysts, in the esterification zone, followed by polycondensation in the prepolymer and finishing zones in the presence of a polycondensation catalyst; or ester exchange usually in the presence of a transesterification catalyst in the ester exchange zone, followed by prepolymerization and finishing in the presence of a polycondensation catalyst, and each may optionally be solid stated according to known methods.

Any one of the following methods for making the composition of the invention can be employed:
(i) the active additives and inactive additives can be added during melt phase manufacture of the base material, such as a polyester polymer, and when withdrawn from the melt phase polycondensation reactor and made into a solid, such as a pellet or bead, that contains the active additives and inactive additives; or
(ii) an amorphous pellet of the base material, such as an amorphous polyester pellet, can be melt blended with the active additives and inactive additives and, after optional crystallization and solid stating, offered as a finally formulated pellet containing the same concentration of the active additives and inactive additives as present in the finished product such that the formulated pellets can be feed through an extruder without a step of separately metering any active additives and inactive additives stream or streams;
(iii) same as method (ii), except that the amorphous pellets are first solid stated after which they are melt blended with the active additives and inactive additives in an extruder to make pellets, the pellets which can later be fed through an extruder to make articles, such as a film or sheet.

The sheet or film may further be transferred to a roll for ease of use, storage, and transport or applied directly to a substrate;
(iv) a salt and pepper blend of base material, the active additives, and the inactive additives, one or both optionally in bead form, pellet form, powder form or ground, that can be blended together in a vessel, melted to form a composition or resin, and the resultant composition or resin is extruded from the vessel and fed to an extruder for forming into an article, such as a film or sheet. Alternatively, the composition may be extruded from the vessel as an extruded noodle that is quenched in an ice/water trough and granulated with a strand cutter; or
(v) the base material, the active additives and the inactive additives can be water based, allowing for wet blending. Methods for wet blending known in the art may be utilized for this embodiment.

In each case, the active additives and inactive additives can be added to the base material, such as a polyester, as a neat stream of active additives and inactive additives or as separate neat streams of active additives and inactive additives, in a suitable liquid carrier, or melt blended with the base material to provide a solid concentrate.

The composition of the present invention ideally has a degree of heat resistance that can withstand high temperatures above 100° F. (54.44° C.), more preferably from about 130° F. (54.44° C.) to about 550° F. (287.78° C.), and most preferably from about 130° F. (54.44° C.) to about 450° F. (232.22° C.) for at least one second without resulting in chemical degradation or functional degradation. The composition is safe for direct food contact.

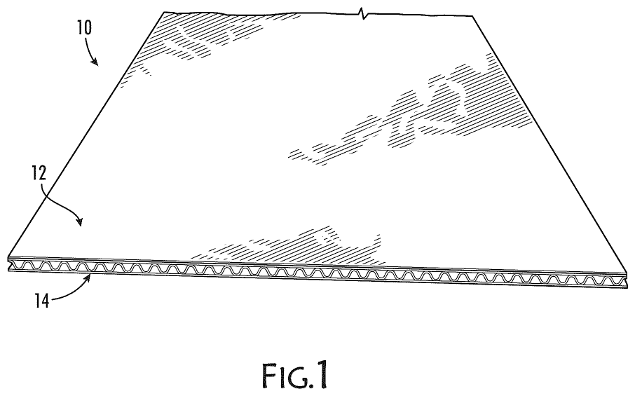

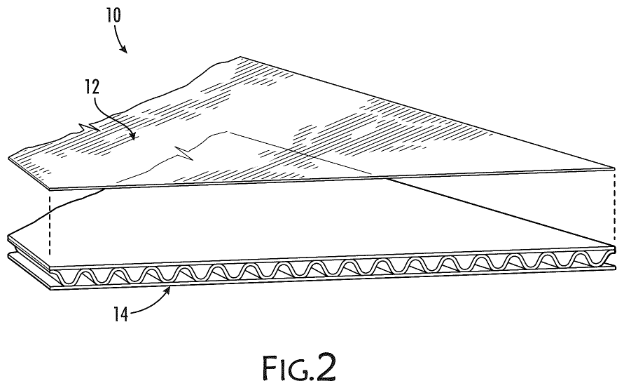

The composition may be formed into an article such as a wrap to package food items, such as produce, and have direct contact with the food item. Alternatively, the composition may be applied to a substrate, forming a layered structure. An exemplary layered structure 10 is illustrated in FIGS. 1 and 2. In this embodiment, the composition is extruded into a film or sheet forming a layer of composition 12, and applied to a substrate 14. The substrate 14 may be paper, such as backing paper, paperboard, a corrugated box, a carton, a tray, a plastic film, a plastic container, or the like. The layered structure 10 may consist of at least one layer of composition 12 and at least one layer of substrate 14, allowing the layered structure 10 to consist of more than one layer of composition 12 and/or more than one layer of substrate. The backing paper utilized in the present invention can range in grammage from about 5 gsm to about 2,000 gsm, and particularly from about 75 gms to about 500 gsm. The combined coatweight of the composition on the substrate is between about 1 gsm to about 150 gms, preferably between about 10 gms to about 50 gsm. The backing paper may be composed of bleached pulp, unbleached pulp, virgin pulp, recycled pulp, or combination thereof. The backing paper may be made with or without additional sizing additives and can be made with or without mill applied coating. In most situations, the backing paper may be processed into a corrugated box, a non-corrugated carton, an insert, a shipping sack, or other like shipping container to store and ship food items, such as produce, and the layer of composition applied to the backing paper will extend the shelf life of the food items. In this embodiment, the composition applied to the substrate will be exposed to the food items stored within the corrugated box, a non-corrugated carton, an insert, a shipping sack, or other like shipping container to scavenge the ethylene produced by the food items within the head space and decreasing the ethylene within the container. The ethylene scavenging agent of the present invention does not block binder sites for ethylene response in food items, such as plants, vegetables, flowers, fruits, and the like.

### Example

An exemplary composition was obtained by mixing pellets of the base material of polypropylene, along with pellets of the ethylene scavenging agent and pellets of the antimicrobial agent in a vessel and blended together, forming a composition. The composition is then heated within the vessel and extruded from the vessel into a sheet. The exact components are set forth in Table 1.

TABLE 1

| | | Ethylene | |
|---|---|---|---|
| | Polypropylene | Scavenging Agent | Antimicrobial Agent |
| Example | wt. % | wt. % | wt. % |
| 1 | 89 | 10 | 1 |

The polypropylene utilized in this example is ExxonMobil™ pp 3155E5, the ethylene scavenging agent is CESAR-absorb 19133 PRA0698710, and the antimicrobial agent is CESA®-am PE J18712-10 PRA0698712.

It should be emphasized that the above-described embodiments of the present disclosure are merely possible examples of implementations set forth for a clear understanding of the principles of the disclosure. Many variations and modifications may be made to the above-described embodiments without departing substantially from the scope and principles of the disclosure. All such modifications and variations are intended to be included herein within the scope of this disclosure and protected by the following claims.

Therefore, the following is claimed:

1. A composition, comprising:
from about 80 wt. % to about 92 wt. % of a base material;
from about 5 wt. % to about 15 wt. % ethylene scavenging agent; and
an antimicrobial agent; and
from about 0.1 wt. % to about 3 wt. % antimicrobial agent, wherein the total wt. % of the composition is 100 wt. %.

2. The composition of claim 1, wherein the base material is a polyester polymer.

3. The composition of claim 1, wherein the ethylene scavenging agent is a thermoplastic concentrate with ethylene scavenging properties.

4. The composition of claim 3, wherein the thermoplastic is polyolefin.

5. The composition of claim 1, wherein the base material is polypropylene.

6. The composition of claim 1, wherein the composition comprises 89 wt. % base material, 10 wt. % ethylene scavenging agent, and 1 wt. % antimicrobial agent.

7. The composition of claim 1, wherein the ethylene scavenging agent is a thermoplastic concentrate with antimicrobial properties.

8. The composition of claim 7, wherein the thermoplastic is a polyolefin.

9. The composition of claim 1, further comprising an inactive additive selected from the group consisting of adhesion promotion agents, minerals, colorants, dyes, plasticizers, chain extenders, impact modifiers, and combinations thereof.

10. An ethylene scavenging composition with antimicrobial properties applied to a substrate, comprising:
at least one layer of a composition, comprising:
a base material;
at least one active additive;
at least one layer of a substrate; and
wherein the layer of composition is applied to a substrate.

11. The ethylene scavenging composition according to claim 10, wherein the active additive may be an ethylene scavenging agent or an antimicrobial agent or both.

12. The ethylene scavenging composition according to claim 10, wherein the base material is a polymer.

13. The ethylene scavenging composition according to claim 10, wherein the active additive is an ethylene scavenging agent that scavenges ethylene from the air surrounding the substrate and does not block binding sites for ethylene response.

14. The ethylene scavenging composition according to claim 10, wherein the substrate is selected from the group consisting of backing paper, paperboard, a corrugated box, a carton, a tray, a plastic film, or a plastic container.

15. The ethylene scavenging composition according to claim 10, wherein the layer of composition is a film or sheet.

16. The ethylene scavenging composition according to claim 10, further comprising an inactive additive selected from the group consisting of adhesion promotion agents, pigments, minerals, colorants, dyes, plasticizers, chain extenders, impact modifiers, and combinations thereof.

17. An ethylene scavenging substrate with antimicrobial properties, comprising:
at least one layer of a substrate; and
at least one layer of a composition, comprising:
a base material;
at least one active additive consisting of an ethylene scavenging agent and an antimicrobial agent;
wherein the at least one layer of composition is applied to a substrate.

18. The ethylene scavenging substrate according to claim 17, with antimicrobial properties, wherein the layer of composition is a sheet or film that is applied to one side of the substrate.

19. The ethylene scavenging substrate according to claim 18, with antimicrobial properties, wherein the substrate is selected from the group consisting of backing paper, paperboard, a corrugated box, a carton, a tray, a plastic film, or a plastic container.

20. The ethylene scavenging substrate according to claim 18, with antimicrobial properties, wherein the one layer of composition comprises: from about 80 wt. % to about 92 wt. % of a base material; from about 5 wt. % to about 15 wt. % of an ethylene scavenging agent; and from about 0.1 wt. % to about 3 wt. % antimicrobial agent, wherein the total wt. % of the composition is 100 wt. %.
